(19) United States
(12) Patent Application Publication
(10) Pub. No.: US 2026/0061153 A1
(43) Pub. Date: Mar. 5, 2026

(54) Kinetic Sleep Apparatus

(71) Applicant: James Ryan, Las Vegas, NV (US)

(72) Inventor: James Ryan, Las Vegas, NV (US)

(21) Appl. No.: 18/820,752

(22) Filed: Aug. 30, 2024

(51) Int. Cl. A61M 21/02; A61M 21/00

(52) U.S. Cl. CPC A61M 21/02; A61M 2021/0022; A61M 2021/0027; A61M 2205/3584

(57) ABSTRACT

A kinetic sleep apparatus that utilizes kinetic energy (vibration) and sound to facilitate relaxation and sleep by a user. The apparatus comprises an amplifier and at least one transducer that can be placed under a mattress, pillow, or seat cushion. A customized audio file is provided which will generate vibration frequencies to simulate the vibrations an individual would experience with an automobile, airplane, cruise ship or even other transportation modes. Each customized audio file includes several other layers in addition to the vibration frequencies, including brain wave frequencies, and natural calming sounds such as rain, ocean waves, wind, didgeridoo, a rainforest, and other soothing sounds of nature.

### FIELD OF INVENTION

The present invention relates to a kinetic sleep aid apparatus which will facilitate a user's ability to fall asleep and enhance the overall sleep process.

### BACKGROUND OF THE INVENTION

Studies have shown that about thirty-four percent (34%) of adults in the United States have some sort of sleep disorder, such as insomnia, which is about fifty million (50,000,000) adults. More significant perhaps, is that about ten percent (10%) of adults in the United States have a sleeping disorder that has a negative impact on their daily lives. Seeking a solution, many sleep-deprived individuals first attempt to change bedding, including mattresses, sheets, pillows. When that type of change does not result in the desired sleep, they look to over-the-counter sleeping aids, including powerful narcotic sleep aids, and more recently, formulations containing essential oils, including cannabis oil. Psychological therapy also has been a sought-after treatment. Often overlooked in sleep therapy is the use of soothing and rhythmic sounds.

### PRIOR ART

The prior art is replete with sleep aid devices. In U.S. Pat. No. 10,434,279 B2 to Keshavan et al., a sleep assistance device is provided which includes a contactless biosensor sensor and a processor to detect a user's sleep state by reading signals from the biometric sensor. The biometric sensor initiates a sleep ready state by playing relaxing sounds or respiration entertainment sound. U.S. Pat. No. 11,431,202 B2 to Ning et al. is directed to a device for assisted sleep comprising a vibration source and/or a sound source which emit vibrations and/or sounds. Ning et al. broadly disclose that the vibration source is or includes a sound which may be integrated as a single device or as two separate devices, particularly for use in a pillow wherein a noise generating device produces white, pink, or brown noise which causes vibrations to stimulate a vagus nerve. However, Ning et al. fails to provide one or more transducers which transfer vibrations kinetically to a mattress while simultaneously transferring audio sounds.

U.S. Patent Application Publication 2006/0205994 A1 to Sunnen discloses an apparatus for generating sleep-inducing stimuli including a programmable controller connected to at least one transducer which generates and applies stimuli including vibration and light. U.S. Patent Application Publication 2017/0281899 A1to Boren provides an infant sleep-inducing device which simulates the vibrations that occur in an automobile. And, WO 2022/021645 A1 to Xie relates to sleep aid devices having blue tooth capability to transmit sleep-aid audio.

Despite the efforts of the prior art, a need still exists for a kinetic sleep aid apparatus which is portable and which includes an amplifier for receiving an audio file, at least one transducer that transmits both audio sounds and vibrations produced solely from the audio file. Such an apparatus should use customized sounds that not only vibrate the bed to the same frequency as an automobile, airplane, or cruise ship, but also layer on top that the brain wave frequencies of beta, alpha, theta, and delta. Preferably, natural sounds can be layered on top of the brain wave frequencies.

### SUMMARY OF THE INVENTION

The invention of the present subject matter relates to a kinetic sleep apparatus which facilitates a user's ability to fall asleep and stay asleep using customized sounds that vibrate the bed to the same frequency as an automobile, airplane, cruise ship, but also layer on top those customized sounds, the beta, alpha, theta, and delta brain wave frequencies and then layer natural sounds on top of the brain wave frequencies.

Accordingly, it is an object of the present subject matter to provide a kinetic sleep apparatus which utilizes customized sounds that can vibrate a bed mattress, pillow, chair, massage table, infant crib, and the like, to simulate the vibration frequencies from an automobile, airplane, or cruise ship.

It is another object of the present subject matter to provide a kinetic sleep apparatus which layers on top of the customized sounds, the beta, alpha, theta, and delta brain wave frequencies.

It is still another object of the present subject matter to provide a kinetic sleep aid apparatus that layers natural soothing sounds on top of the brain wave frequencies.

It is a yet another object of the present subject matter to provide a kinetic sleep apparatus that enhances the overall sleep process by the combination of auditory and vibratory sensations from these customized sounds, brain wave frequencies, and natural sounds.

It is a further object of the present subject matter to provide a kinetic sleep apparatus which allows for multiple variations of vibrations including the ability to overlap binaural frequencies, FM radio, or other sounds.

It is yet a further object of the present subject matter to provide a kinetic sleep apparatus that is portable such that it can be used during travel.

It is still a further object of the present subject matter to provide a kinetic sleep apparatus having at least one transducer that can be positioned easily between the box spring and mattress of a bed and directed to a specific area and can be adjusted quickly for different sides of a bed.

It is another object of the present subject matter to provide a kinetic sleep apparatus having up to four transducers, each which can be position between a box spring and mattress of a bed to generate vibrations to specific areas of the bed.

It is an additional object of the present subject matter to provide a kinetic sleep apparatus that can be utilized with massage beds, chairs, and cribs

It is another object of the present invention to provide a kinetic sleep apparatus that can be used to facilitate a user's ability to fall asleep without the use of over-the-counter drugs or other narcotic sleep aids.

These and other objects of the present subject matter are accomplished by providing a kinetic sleep apparatus comprising an amplifier having at least one audio file input, a bluetooth adapter, and an output for each transducer, at least one transducer that generates vibrations which transfer kinetic energy to the user's body and produces sound to the user's ears, at least one audio file which is removably engaged in the audio file input of the amplifier, and a power supply. Preferably, the kinetic sleep apparatus further comprises a remote-control device and carrying case.

### BRIEF DESCRIPTION OF DRAWINGS

FIG. 1 is perspective view of each of the elements of the kinetic sleep apparatus of the present subject matter.

FIG. 2 is top perspective view of the kinetic sleep apparatus of the present subject matter disposed within a carrying case.

### DETAILED DESCRIPTION OF THE INVENTION

In the following description, like reference numbers are used to identify like elements. Furthermore, the drawings are intended to illustrate major features of exemplary embodiments in a diagrammatic manner. The drawings are not intended to depict every feature of every implementation nor relative dimensions of the depicted elements and are not drawn to scale. In the following description, numerous specific details are set forth to clearly describe various specific embodiments disclosed herein. One skilled in the art, however, will understand that the presently claimed invention may be practiced without all the specific details discussed below. In other instances, well known features have not been described so as not to obscure the invention.

The terms “sleeping device”, “sleep aid device, “sleep machine”, and similar terms may be used throughout the specification and it is to be understood that such terms are interchangeable and all refer to the kinetic sleep apparatus of the present subject matter.

Also, it is to be understood that the phraseology and terminology used herein is for the purpose of description and should not be regarded as limiting. The use of the terms “including”, “comprising”, “having”, and variations thereof herein is meant to encompass the items listed thereafter and equivalents thereof as well as additional items.

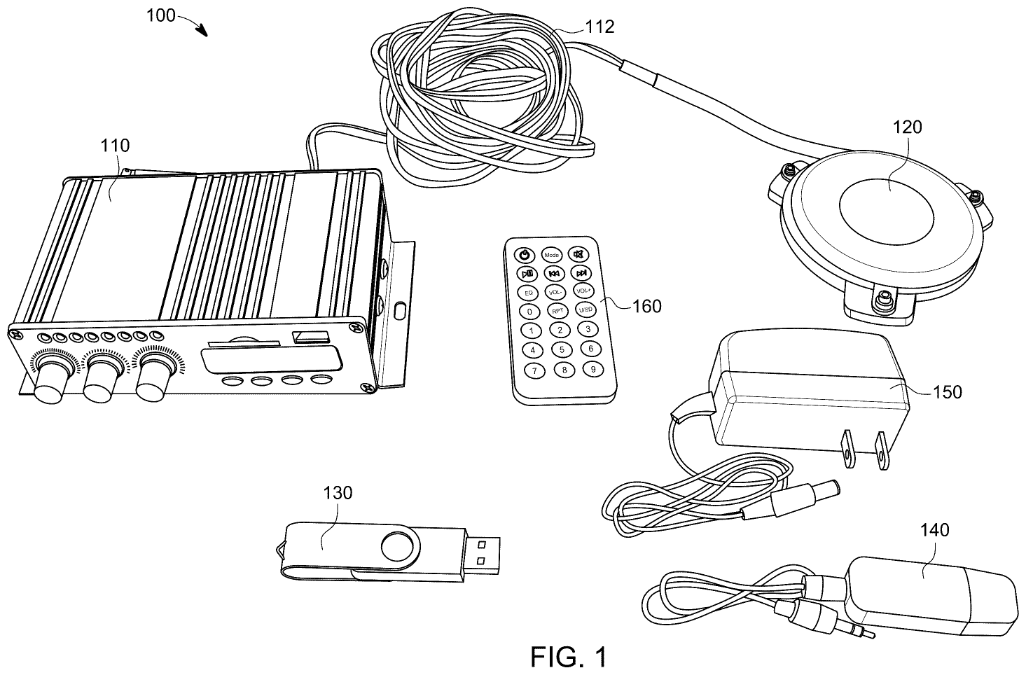

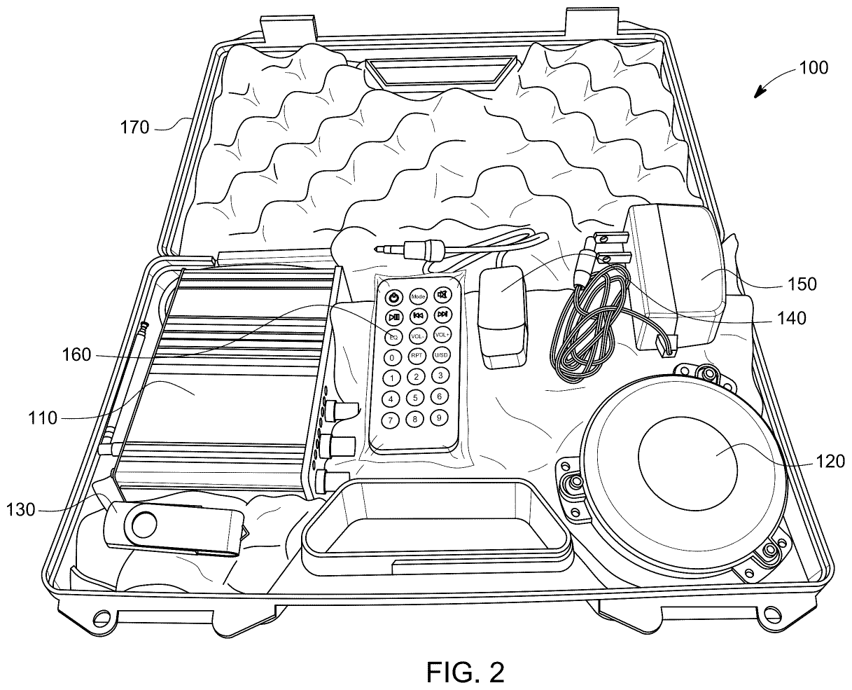

The kinetic sleep apparatus of the present subject matter is portable and can be transported easily for travel in a carrying case. Referring to FIG. 1, the kinetic sleep apparatus 100 comprises an amplifier 110, at least one transducer 120, at least one audio file 130, a bluetooth device 140 and a power adapter 150 for connecting to a power source. Preferably, the kinetic sleep apparatus further comprises a remote-control device 160 and a carrying case 170 as illustrated in FIG. 2.

The amplifier 110 is the “brain” of the kinetic sleep apparatus of the present subject matter. It is of a sufficiently small size such that it can be placed on a night stand. The amplifier has several inputs for audio files 130, such as a micro-SD card port, USB port, a standard 3.5 mm audio jack port, and ⅛^{th} inch mini-jack port. The amplifier also has a plurality of outputs capable of connecting to a plurality of transducers 120. Amplifier 110 also has an input for connecting to a power adapter 150 and a bluetooth adapter for connecting to a blue tooth device 160. The amplifier also is provided with left and right control knobs, base and treble adjusting knobs, and a volume adjustment knob.

The transducer 130 is in the form of a small, hockey puck-shaped device that can be placed between the mattress and box spring of a bed, under a bed, under a pillow, behind a pillow, under a seat cushion, and any other medium and placement depending upon the application of use. Each transducer is removably connected to one of the plurality of outputs on the amplifier 110 by a small wire 112, such as a conventional insulated wire which will be obvious to those skilled in the art. In one embodiment, there is a single transducer 130. Preferably, the kinetic sleep apparatus comprises a plurality of transducers, each separately and removably connected to an amplifier output. More preferably, the plurality of transducers is up to four transducers each removably connected to one of four amplifier outputs. The transducer simultaneously vibrates and produces sound from the audio file. The resulting vibration transfers that kinetic energy through the mattress, pillow or other medium to the user's body while the sound is received aurally.

The customized audio file can be provided in any suitable format which will be obvious to those skilled in the art, suitable examples of which can be a USB device, SD-card, Bluetooth, a smart electronic device such as a smart phone or smart tablet, and similar formats. The audio file is customized to be used with the kinetic sleep apparatus and will generate vibration frequencies to simulate the vibrations an individual would experience with an automobile, airplane, cruise ship or other transportation modes. Each customized audio file can include several other layers in addition to the simulated vibration frequencies including brain wave frequencies and natural sounds such as rain, ocean waves, wind, and the like. The customized audio file may further comprise vibrations having the ability to overlap binaural frequencies, FM radio frequencies and other sounds. It is contemplated that audio files can be customized via a website to meet the specific desires and needs of the user. The audio file can be downloaded from the website or it can be programmed onto a thumb drive, USB device or SD card and sent to the user.

The incorporation of a layer of brain wave frequencies is significant in facilitating sleep to the user. As is generally known, the brain is an electrochemical organ which emits four kinds of brain waves, the first being beta waves, having relatively low amplitude and a frequency ranging from between about 15 to about 40 cycles per second. The second type of brain wave frequency is alpha, ranging from between about 9 to about 14 cycles per second. The third kind of brain wave is theta, having a frequency ranging from between about 5 and about 14 cycles per second. The fourth type of brain wave frequency is delta frequency, ranging from between about 1.5 to about 4 cycles per second. The delta brain wave frequency is the slowest and has the greatest amplitude. In deep sleep, the delta brain wave frequency can be between about 2 to about 3 cycles per second. When an individual is ready to sleep, the individual typically is in a low beta frequency. As the onset of sleep proceeds, the user's brain waves decrease from beta, to alpha, to theta and then to delta when the individual falls asleep.

The kinetic sleep apparatus of the present subject matter further comprises a bluetooth device 140 which is removably connected to the amplifier via the bluetooth adapter. The addition of the bluetooth device will enable a smart phone or other bluetooth-enabled device to play the customized audio file.

The kinetic sleep apparatus 110 can be powered by any suitable power source including an electric power source or a rechargeable battery adapter or battery pack. Preferably, the power source is a simple power adapter 150 which can be plugged into an electrical outlet. In a preferred embodiment, the kinetic sleep apparatus further comprises a remote-control device which enables the user to control the amplifier from anywhere within a predefined space or room.

The kinetic sleep apparatus 100 of the present subject matter is portable and can be quickly and easily set up by the user. To facilitate transporting the apparatus, particularly during travel, a carrying case 170 is provided as illustrated in FIG. 2. In this manner, the kinetic sleep apparatus can be used in a hotel room, the residence of a family member or friend, modes of transportation, health spas, massage tables, and day beds. It also can be used with a couch, chair, office chair or infant crib. Preferably, the carrying case is also the shipping case.

While several illustrative embodiments of the invention have been shown and described, numerous variations and alternative embodiments will occur to those skilled in the art. Such variations and alternative embodiments are contemplated and can be made without departing from the scope of the invention as defined in the appended claims.

1. A kinetic sleep apparatus comprising:
a. an amplifier having a plurality of inputs for receiving audio files,
a plurality of outputs, each output capable of being removably connected to a transducer, a bluetooth adapter and a power source adapter;
b. a plurality of transducers, each transducer being removably connected to one o said plurality of outputs, each of said plurality of transducers generating vibrations kinetically while simultaneously transferring audio sounds;
c. a customized audio file which generates vibration frequencies to simulate the vibrations an individual experiences in a transportation device and further generating additional frequencies layers including brain wave frequencies and natural sounds;
d. a bluetooth device which can be removably connected to said bluetooth adapter, and
e. a power adapter for connecting to a power source.

2. The kinetic sleep apparatus in accordance with claim 1, further comprising a remote control for activating and deactivating the kinetic sleep apparatus.

3. The kinetic sleep apparatus in accordance with claim 1, further comprising a carrying case.

4. The kinetic sleep apparatus in accordance with claim 1, wherein said bluetooth device provides a wireless connection to a smart device, selected from the group consisting of a smart phone, a smart tablet, and a smart watch.

5. The kinetic sleep apparatus in accordance with claim 1, wherein said amplifier is of a sufficiently small size such that it can be placed on a night stand.

6. The kinetic sleep apparatus in accordance with claim 1, wherein said plurality of audio inputs include a micro-SD card port, a USB port, a standard 3.5 audio jack port, and a ⅛^{th} inch mini-jack port.

7. The kinetic sleep apparatus in accordance with claim 1, wherein said plurality of transducers is one transducer.

8. The kinetic sleep apparatus in accordance with claim 1, wherein said plurality of transducers is four transducers.

9. The kinetic sleep apparatus in accordance with claim 1, wherein each of said transducers is in the form of a hockey-puck shaped and sized device.

10. The kinetic sleep apparatus in accordance with claim 1, wherein said customized audio file is programmed onto a device selected from the group consisting of a USB drive, a micro-SD card, a bluetooth device, and a smart electronic device.

11. The kinetic sleep apparatus in accordance with claim 1, wherein said customized audio file is downloaded onto a device selected from the group consisting of a USB drive, a micro-SD card, a bluetooth device, and a smart electronic device.

12. The kinetic sleep apparatus in accordance with claim 1, wherein said power source adapter can be connected to a power source selected from the group consisting of an electric power source and a battery powered source.

13. The kinetic sleep apparatus in accordance with claim 1, wherein said customized audio file generates vibration frequencies to simulate vibrations from a transportation device selected from the group consisting of an automobile, an airplane, and a ship.

14. The kinetic sleep apparatus in accordance with claim 13, wherein said customized audio file is layered further with beta, alpha, theta, and delta brain wave frequencies.

15. The kinetic sleep apparatus in accordance with claim 14, wherein said customized audio file is layered further with at least one natural sound selected from the group consisting of rain, ocean waves and wind.

16. The kinetic sleep apparatus in accordance with claim 13, wherein said customized audio file further comprises vibrations having the ability to overlap binaural frequencies and FM radio frequencies.

17. A kinetic sleep aid in accordance comprising:
a. an amplifier being of a sufficiently small sized to rest on a nightstand, said amplifier comprising a plurality of inputs for receiving audio files, a plurality of outputs, each outlet capable of being removably connected to a transducer;
b. at least one transducer in the form of a small circular shaped device that can be disposed between a mattress and box spring, each of the at least one transducer being provided with a small wire which can be removably connected to one of said plurality of outputs, each of the at least one transducer capable of generating vibrations and simultaneously producing sound, the vibrations transferring kinetic energy through said mattress to a body lying on said mattress;
c. a customized audio file which generates vibration frequencies to simulate the vibrations an individual experiences in a transportation device and further generating additional frequencies layers including brain wave frequencies and natural sounds;
d. a bluetooth device which can be removably connected to said bluetooth adapter;
e. a power adapter for connecting to a power source;
f. a remote-control device, and
g. a carrying case capable of retaining and safely transporting said kinetic sleep apparatus.
